(19) United States
(12) Patent Application Publication
(10) Pub. No.: US 2010/0210899 A1
(43) Pub. Date: Aug. 19, 2010

(54) Method for percutaneous lateral access to the left ventricle for treatment of mitral insufficiency by papillary muscle alignment

(75) Inventor: Kemal Schankereli, Stillwater, MN (US)

Correspondence Address: JUNEAU PARTNERS, 333 N Fairfax St., Suite 305, ALEXANDRIA, VA, 22301, US

(73) Assignee: TENDYNE MEDICAL, INC.

(21) Appl. No.: 12/749,487

(22) Filed: Mar. 29, 2010

Related U.S. Application Data
(63) Continuation-in-part of application No. 12/691,591, filed on Jan. 21, 2010.
(60) Provisional application No. 61/146,144, filed on Jan. 21, 2009.

(51) Int. Cl. A61F 2/00

(52) U.S. Cl. 600/37; 128/898

(57) ABSTRACT

This invention relates to devices and methods for the therapeutic changing of the geometry of the left ventricle of the human heart. Specifically, the invention relates to the left-ventricular lateral wall introduction of an anchoring device to align the papillary muscles.

### CROSS REFERENCE TO RELATED APPLICATIONS

This application is a continuation-in-part and claims priority under 35 USC 120 to U.S. Ser. No. 12/691,591, filed 21 Jan. 2010, entitled Apical Papillary Muscle Attachment for Left Ventricular Reduction, the contents of which are incorporated by reference herein in their entirety, which claims priority benefit under 35 USC 119(e) to U.S. 61/146,144, filed 21 Jan. 2009.

### STATEMENT REGARDING FEDERALLY SPONSORED RESEARCH OR DEVELOPMENT

No federal government funds were used in researching or developing this invention.

### NAMES OF PARTIES TO A JOINT RESEARCH AGREEMENT

n/a

### REFERENCE TO A SEQUENCE LISTING

n/a.

### BACKGROUND

1. Field of the Invention

This invention relates to devices and methods for the therapeutic changing of the geometry of the left ventricle of the human heart. Specifically, the invention relates to the percutaneous lateral introduction of an anchoring device to align the papillary muscles.

2. Background of the Invention

According to the Center for Disease Control, heart disease is the leading cause of death in the United States and is a major cause of disability. Almost 700,000 people die of heart disease in the U.S. each year. That is about 29% of all U.S. deaths. Heart disease is a term that includes several more specific heart conditions.

One of these conditions is cardiomyopathy. Cardiomyopathy is a weakening of the heart muscle or a change in heart muscle structure. It often results in inadequate heart pumping or other heart function abnormalities. These can result from various causes, including prior heart attacks, viral or bacterial infections, and others.

The geometry of the myocardium is critical to proper functioning. The myocardium is comprised of a single, continuous tissue that wraps around itself, spiraling up from the apex of the heart, to form a helix with elliptically shaped ventricles. This spiral produces an oblique muscle fiber orientation, meaning that the fibers form a more ventricle ‘x’ shape, so that when fibers shorten 15%, it produces a 60% ejection fraction. Because of its elliptical shape and defined apex, the ventricle is subjected to a relatively low level of lateral stress.

However, a dilated left ventricle is generally due to the effects of a myocardial infarction. An occlusion, or blockage, of cardiac arteries results in either an akinetic (non-beating) or dyskinetic (irregular beating) tissue downstream from the occlusion. This downstream ventricular tissue is damaged, but since the volume of blood that fills the ventricle does not change, the damaged organ has to work harder to eject the blood. This increased load causes an increase in the radius of the ventricle and the thickness of the ventricular wall changes. Further, the apex of the heart becomes circular, the remaining myocardial tissue suffers from pathological hypertrophy, and the valve opening widens. As the ventricle dilates, the muscle fiber orientation, which is critical to a good ejection fraction, becomes transverse, or more horizontal. Subsequently, the ejection fraction decreases; a 15% shortening of muscle fibers now produces only a 30% ejection fraction. The lateral stress on the ventricle increases. Overall, the dilated left ventricle cannot produce a strong enough pulse to maintain health and efficient circulatory return.

Ventricular reduction is a well-known type of operation in cardiac surgery to reduce enlargement of the heart from cardiomyopathy. In 1985, Vincent Dor, Md., introduced endoventricular circular patch plasty (EVCPP), or the Dor procedure, as a viable method for restoring a dilated left ventricle to its normal, elliptical geometry. The Dor procedure, which uses a circular suture and a Dacron® patch to correct LV aneurysms and exclude scarred parts of the septum and ventricular wall, has been one option for ventricular remodeling. The procedure restores ventricular shape, increases ejection fraction, decreases the left ventricular end systolic volume index (LVESVI), and allows for complete coronary revascularization.

The disadvantage to the Dor procedure is that it places synthetic tissue inside the LV cavity and it is usually done as part of a coronary artery bypass graft (open heart) surgery.

Others have attempted further solutions to this problem. U.S. Pat. No. 7,060,021 to Wilk discloses a type clamp for the left ventricle which pulls opposing walls of the heart together in order to close off lower portions of both ventricles.

U.S. published patent application 2007/0083076 to Lichtenstein discloses methods and devices for altering the blood flow through the left ventricle by engaging the outer surface of the heart in a type of binding.

U.S. published patent application 2008/0293996 to Evans discloses a system and method for volume reduction by inserting a conical polymeric container, i.e. balloon, into the left ventricle to reduce the volume of blood flow.

Additionally, many patents and publications are directed to the catheter based repair of the mitral valve using various types of sutures and tethers. For example, U.S. published patent application 2008/0243150 to Starksen discloses a valve annulus treatment device secured by anchors that cinch or draw together circumferentially to tighten the valve annulus (ring). Starksen also discloses that such a device can be delivered by advancing a catheter through the aorta. Published PCT patent application WO/2006/135536 to De Marchena discloses a papillary muscle tether for left ventricular reduction by delivery either (1) through the femoral vein and delivered to the left ventricle via a trans-septal approach into the left atrium, across the mitral valve, or (2) retrograde through the femoral artery, advanced through the aortic valve, and into the left ventricle. However, cardiac catheterization poses the risk of blood clots that can trigger strokes, damage to blood vessels, and damage to the heart or pericardium. Thus, procedures and devices which address these and other concerns are needed in the field.

### BRIEF SUMMARY OF THE INVENTION

Accordingly, in a preferred embodiment of the invention, there is provided a method for improving cardiac function, comprising the steps of: inserting by percutaneous approach a tether installation device into a patient; and inserting said tether installation device through the lateral wall of the left ventricle of the patient's heart; attaching at least one tethered papillary muscle anchor from within said tether installation device to a papillary muscle within said left ventricle; withdrawing said tether installation device from said left ventricle such that the tether of the tethered papillary muscle anchor tranverses the left ventricular wall and extends from the inside to the outside of the left ventricle; attaching a pledget to the tether portion outside the left ventricle to form a wall anchor; wherein said papillary anchor and said wall anchor are joined by the tether to change the geometry and reduce the volume of the left ventricle.

In another preferred embodiment of the invention, there is provided a method for reducing ventricular volume, comprising the steps of : inserting by percutaneous approach a tether installation device into a patient; and inserting said tether installation device through the lateral wall of the left ventricle of the patient's heart and into the left ventricle of the patient's heart; and attaching a first tethered papillary muscle anchor from within said tether installation device to a papillary muscle within said left ventricle; attaching a second tethered papillary muscle anchor from within said tether installation device to a second papillary muscle of the left ventricle of the patient's heart; withdrawing said tether installation device from said left ventricle such that the tethers of the tethered papillary muscle anchors tranverse the left ventricular wall and extend from the inside to the outside of the left ventricle; attaching one or more pledgets to the tether portions outside the left ventricle to form one or more wall anchors; wherein said papillary anchors and said wall anchor are joined by the tethers to change the geometry and reduce the volume of the left ventricle.

In another preferred embodiment of the invention, there is provided a method as described herein further comprising the step of adjusting the length of the tethers to achieve a desired geometry of the left ventricle.

In another preferred embodiment of the invention, there is provided a method as described herein further comprising the steps of attaching at least one additional tethered papillary anchor joined by an additional tether so as to achieve a desired geometry of the left ventricle.

In another preferred embodiment of the invention, the method comprises the steps of piercing the posterior papillary muscle, extending the catheter to install one or more tethered anchors to the anterior papillary muscle, withdrawing the catheter to install one or more tethered anchors onto the posterior papillary muscle, and then cinching the tethers together to achieve the desired geometry of the left ventricle and/or the mitral valve.

In another embodiment, the posterior papillary is pierced, and the anterior papillary is captured by looping one or more tethers around it, the tether(s) is/are tightened to cinch the papillary muscles are cinched without use of anchors, or with minimal use of anchors only on the posterior papillary.

In yet another preferred embodiment of the invention, the method comprises the steps of piercing the cardiac septum, extending the catheter to install one or more tethered anchors to the posterior papillary muscle, withdrawing the catheter to install one or more tethered anchors onto the septum, then cinching the tethers together to achieve the desired geometry of the left ventricle and/or the mitral valve.

In another embodiment, the septum is pierced, and the posterior papillary is captured by looping one or more tethers around it, the tether(s) is/are tightened to cinch the papillary muscle without use of anchors, or with minimal use of anchors only on the septum.

In yet another preferred embodiment of the invention, the method comprises the steps of piercing the left ventricular wall, extending the catheter to install one or more tethered anchors to the posterior papillary muscle and/or the anterior papillary muscle, withdrawing the catheter to install one or more tethered anchors onto the left ventricular wall, then cinching the tethers together to achieve the desired geometry of the left ventricle and/or the mitral valve.

In another embodiment, the left ventricular wall is pierced, and the posterior and/or anterior papillary is captured by looping one or more tethers around it, the tether(s) is/are tightened to cinch the papillary muscle without use of anchors, or with minimal use of anchors only on the left ventricular wall.

In another preferred embodiment of the invention, there is provided a method as described herein further comprising the step of adjusting the tether member to achieve coaptation of the mitral valve.

In another preferred embodiment of the invention, there is provided a method as described herein further comprising wherein the inserting of said tether device includes passing said tether device through a trocar sleeve or canula.

In another preferred embodiment of the invention, there is provided a method as described herein further comprising where inserting the tether device into a patient is performed by percutaneously inserting a needle or trocar having a catheter into the patient through the intercostal space of the patient or by subxyphoid introduction.

In another preferred embodiment, the needle is a non-coring needle to reduce defect to the tissue(s). Preferably, the needle used is a small gauge needle used as a guide to reduce the defect made to the tissue, and the opening is then temporarily dilated using the larger bore instrument to house the catheter.

In another preferred embodiment of the invention, there is provided a method as described herein further comprising implanting a hemostasis valve at the lateral wall insertion site on the heart of the patient, wherein said valve is a blood leakage control valve/sleeve.

In another preferred embodiment of the invention, there is provided a medical device for improving cardiac function or reducing ventricular volume, comprising: a canula having a tethering device disposed therein; said canula having a trocar or needle for percutaneously accessing the chest cavity by intercostal or subxyphoid introduction, said trocar or needle capable of piercing the lateral wall of the left ventricle of the patient's heart and a leakage control hemostasis valve/sleeve; said tethering device comprising at least one first papillary muscle anchor for attaching to a first papillary muscle within said left ventricle and at least one second papillary muscle anchor for attaching to the second papillary muscle of the left ventricle of the patient's heart; said tethering device further comprising a tether member for joining said first papillary muscle anchor to said second papillary muscle anchor so as to reduce the left ventricular volume of the patient.

In another preferred embodiment of the invention, there is provided a device as described herein wherein said tether member has an adjustable mechanism for adjusting the length of said tether.

In another preferred embodiment of the invention, there is provided a device as described herein further comprising at least one additional papillary anchor joined by an additional tether member so as to achieve a desired geometry of the left ventricle.

In another preferred embodiment of the invention, there is provided a device as described herein wherein the tether member is comprised of nitinol (nickel-titanium shape memory alloy) or austinetic stainless steel.

### BRIEF DESCRIPTION OF THE DRAWINGS

FIG. 1A is a graphical representation of a lateral-approach introduction device used to align papillary muscles. FIG. 1A shows canula and the tethering member with four protruding anchors and depth gauge.

FIG. 1B is a graphical representation of a lateral introduction device used to align papillary muscles. FIG. 1B shows canula and the tethering member with three protruding anchors and depth gauge.

FIG. 1C is a graphical representation of a lateral introduction device used to align papillary muscles. FIG. 1C shows canula and the tethering member with one protruding anchor and depth gauge.

FIG. 2 is a drawing of a heart having an enlarged left ventricle.

FIG. 3 is a drawing of a patient in cross-section with a catheter device shown accessing the heart via a chest wall hub.

FIG. 4 is a drawing of a lateral introduction device/catheter percutaneously accessing the heart via the 5th intercostal space with catheter entering the ventricular wall and piercing the anterior papillary.

FIG. 5 is a drawing of a lateral introduction device/catheter percutaneously accessing the heart via the 5th intercostal space with catheter entering through the ventricular wall without piercing the anterior papillary.

FIG. 6 is a drawing of a lateral introduction device/catheter percutaneously accessing the left ventricle of the heart via the 5th intercostal space of a patient's right side with catheter entering through a trans-septal opening from the right atrium to the left atrium for a trans-septal approach.

FIG. 7A is a drawing illustrating a three-shafted percutaneous access instrument. FIG. 7B is a drawing illustrating a tip of the tethering tool and shows an external canula with a spring-loaded pointed obdurator sheathed inside of it, and an inner sleeve containing a butterfly anchor and tether, which is optionally coiled to assist installation of the tethered anchor.

FIG. 8 is a drawing of a heart and left ventricle having a tethered anchor implanted in the posterior papillary muscle.

FIG. 9 is a drawing of a heart showing both a tethered anchor implanted in the anterior papillary muscle and a tethered anchor previously anchored in the posterior papillary muscle resulting in two papillary muscles anchored with an unadjusted, or loose, tether.

FIG. 10 is a drawing of an anterior and posterior papillary muscle having a tether inserted through an opening in the muscle tissue, and shows a figure-8, or butterfly, anchor attached to the end of the tether and located on the distal side of the posterior papillary and a cinching disk attached to the tether and located on the proximal side of the anterior papillary.

FIG. 11A is a drawing of a metallic cinch, or cinching, disk attached to a tether, where the disk has an aperture through which the tether runs, and shows Step 1 of a process of cinching, or clamping the cinch disk onto the tether at a specific location of the tether equal to a desired length of tether. FIG. 11B is a drawing of the cinch disk on the tether and shows Step 2 of the cinching process where the cinch disk is crimped or folded 180 degrees. FIG. 11C is a drawing of the cinch disk on the tether and shows final Step 3 of the cinching process where the halfed-cinch disk is again crimped or folded a second 180 degrees created a locked cinch disk, wedge shaped from the crimping, locked in place at that location on the tether.

FIG. 12 is a drawing of a corrected heart showing the tethers gathered by an adjustable connector.

FIG. 13 is a drawing of a corrected heart showing a circular tether embodiment.

### DETAILED DESCRIPTION OF THE INVENTION

### Definitions

The following definitions are provided as an aid to understanding the detailed description of the present invention.

“Anchors” for the purposes of this application, is defined to mean any fastener. Thus, anchors may comprise C-shaped or semicircular hooks, curved hooks of other shapes, straight hooks, barbed hooks, clips of any kind, T-tags, or any other suitable fastener(s). In one embodiment, anchors may comprise two tips that curve in opposite directions upon deployment, forming two intersecting semi-circles, circles, ovals, helices or the like. In some embodiments, anchors are self-deforming. By “self-deforming” it is meant that anchors change from a first undeployed shape to a second deployed shape upon release of anchors from restraint in housing. Such self-deforming anchors may change shape as they are released from housing and enter papillary or myocardial tissue, to secure themselves to the tissue. Thus, a crimping device or other similar mechanism is not required on distal end to apply force to anchors to attach them to tissue.

Self-deforming anchors may be made of any suitable material, such as a super-elastic or shape-memory material like Nitinol or spring stainless steel. In other embodiments, anchors may be made of a non-shape-memory material and made be loaded into housing in such a way that they change shape upon release. Alternatively, anchors that are not self-deforming may be used, and such anchors may be secured to tissue via crimping, firing or the like. Even self-securing anchors may be crimped in some embodiments, to provide enhanced attachment to tissue. In some embodiments, anchors may comprise one or more bioactive agent. In another embodiment, anchors may comprise electrodes. Such electrodes, for example, may sense various parameters, such as but not limited to impedance, temperature and electrical signals. In other embodiments, such electrodes may be used to supply energy to tissue at ablation or sub-ablation amounts. Delivery of anchors may be accomplished by any suitable device and technique, such as by simply releasing the anchors. Any number, size and shape of anchors may be included in housing.

Lateral approach or the lateral wall of the left ventricle, refers to accessing a known part of the heart, roughly equivalent to the somewhat planar, muscular side wall of the organ, between the bottom of the heart and the left atrium.

Canula or canula refers to a well-known tube-like medical instrument. It can be fitted with a trocar or needle, a sharp pointed device for piercing tissue. In one preferred embodiment, the needle or trocar is a non-coring needle or trocar to reduce defect to the tissue being pierced. Preferably, the needle used is a small gauge needle used as a guide to reduce the defect made to the tissue, and the opening is then temporarily dilated using the larger bore instrument to house the catheter.

Tether may be one long piece of material or two or more pieces and may comprise any suitable material, such as Nitinol, austinetic steel, suture, suture-like material, a Dacron strip or the like.

Hemostasis valve, or valve/sleeve, refers to a device which allows the heart tissue to be pierced at the lateral wall region with little or no blood loss. Similar valves/sleeves are well known in the venipuncture field where individual vacutainers can be repeatedly mounted on a single needle, and valves such as the Touehy Borst valve which allows multiple insertions of catheters while maintaining hemostasis.

Percutaneous or percutaneous approach refers to pertains to a medical procedure where access to inner organs or other tissue, in this case the pericardium and heart through the chest cavity, is done via trocar or needle-puncture of the skin, rather than by using an “open” approach where inner organs or tissue are exposed (typically with the use of a scalpel). In preferred embodiments, the heart is accessed through the intercostal space, or alternatively by subxyphoid introduction to the chest cavity.

Generally, delivery of the tether device may be advanced by any suitable advancing or device placement method so long as it arrives at the lateral wall of the left ventricle of the heart. Many catheter-based, minimally invasive devices and methods for performing intravascular procedures, for example, are well known, and any such devices and methods, as well as any other devices or method later developed, may be used to advance or position delivery device into a desired location. For example, in one embodiment a steerable guide catheter is first advanced percutaneously to the lateral wall region. In a preferred embodiment, catheter is advanced through the intercostal space, more preferably between the 4th, 5th or 6th intercostal space. In another embodiment, the catheter is advanced via subxyphoid access to the normal pericardium. The steerable catheter is inserted into the left ventricle of the heart through the lateral wall of the left ventricle and thus into the space formed by left ventricle. An obturator pushes or holds the tissue in place once it has been pierced. Once in this space, the steerable catheter is easily advanced to the papillary muscle or to the ventricular wall, one or more anchors may then be advanced and attached to/inserted into the papillary muscle and/or the LV myocardium and/or the septum. Of course, this is but one exemplary method and any other suitable method, combination of devices, etc. may be used.

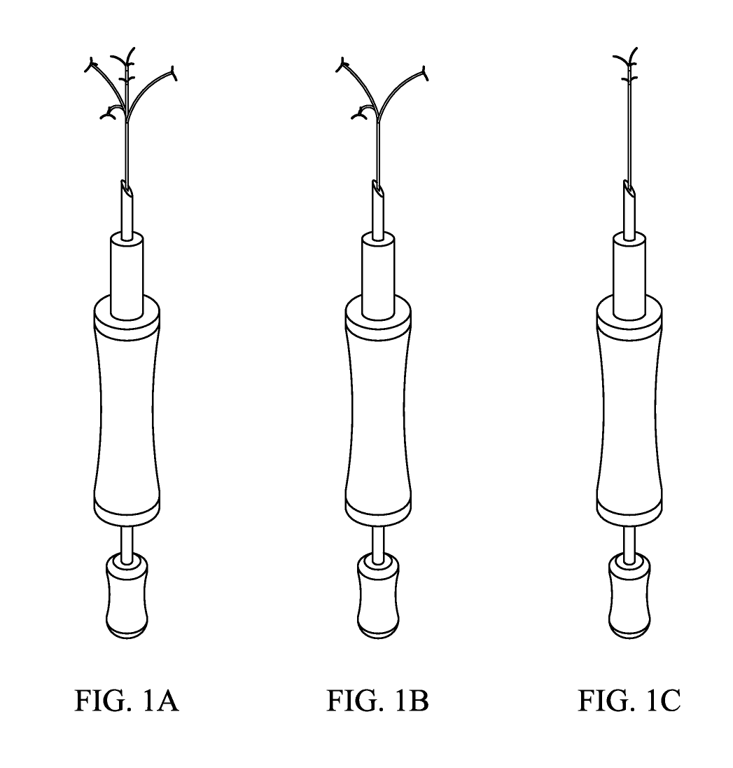

Referring now to the figures, FIG. 1A is a graphical representation of a lateral-approach introduction device used to align papillary muscles. FIG. 1A shows canula and the tethering member with four protruding anchors and depth gauge.

The novel introduction instrument described herein for percutaneous intercostal penetration includes a handle having a two-spring mechanism, a uniquely curved tip, and a triple shaft. In practice, a 5-6 cm cut is made for access to the heart. After passing through the 5th intercostal space, the pericardium is cut, exposing the epicardial surface of the heart. Using imaging to find the papillary muscles of the left ventricle, the instrument functions as a dilator to dilate the way into the heart. This is an important structural and functional difference since the heart muscle itself does not suffer the coring of the tissue that has been observed in previous techniques. Once a working port is established, imaging may again be used to confirm the location of the papillary muscles. Using a penetrating instrument such as a needle, the anterior papillary is, in one preferred embodiment, pierced and the tool is advanced across the left ventricular space to the posterior papillary. Using a spring-loaded fine canula, an anchor is pushed through the posterior papillary. In a preferred embodiment, the anchor is figure-8 shaped such that, while stored within the canula, the anchor is compressed, but upon spring injection into the posterior papillary, the figure-8 opens and expands on the distal side of the papillary to form a barrier anchor. In another preferred embodiment, a fish-hook style anchor is contemplated. Once the posterior papillary has been anchored, and the tether extends back through the aperture of the pierced anterior papillary, a cinch or snugger part is advanced down the tether toward the proximal side of the anterior papillary. By pulling the tether, the papillary muscles are drawn together, and the cinch, or snugger, part may be used to lock the papillary distance in place by securing the tether at a specific length.

FIG. 1B is a graphical representation of a lateral introduction device used to align papillary muscles. FIG. 1B shows canula and the tethering member with three protruding anchors and depth gauge.

FIG. 1C is a graphical representation of a lateral introduction device used to align papillary muscles. FIG. 1C shows canula and the tethering member with one protruding anchor and depth gauge.

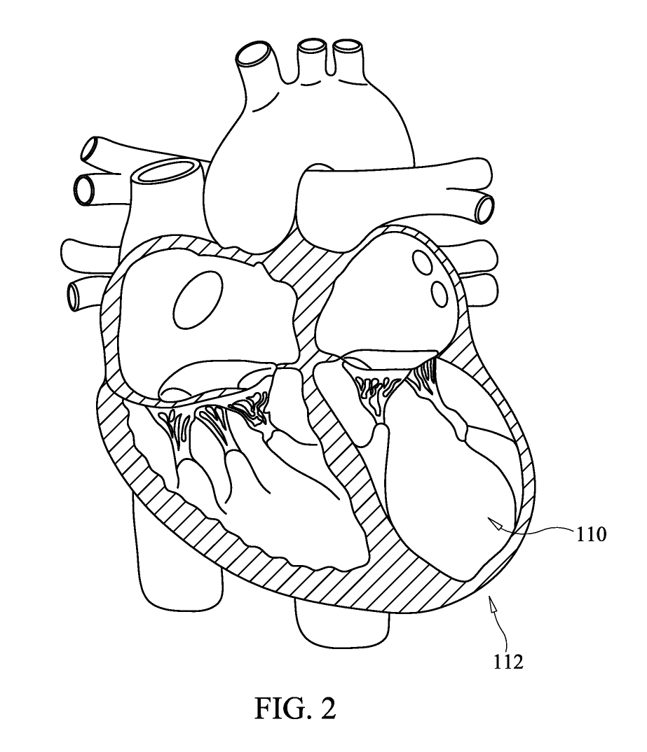

FIG. 2 is a drawing of a heart 112 having an enlarged left ventricle 110.

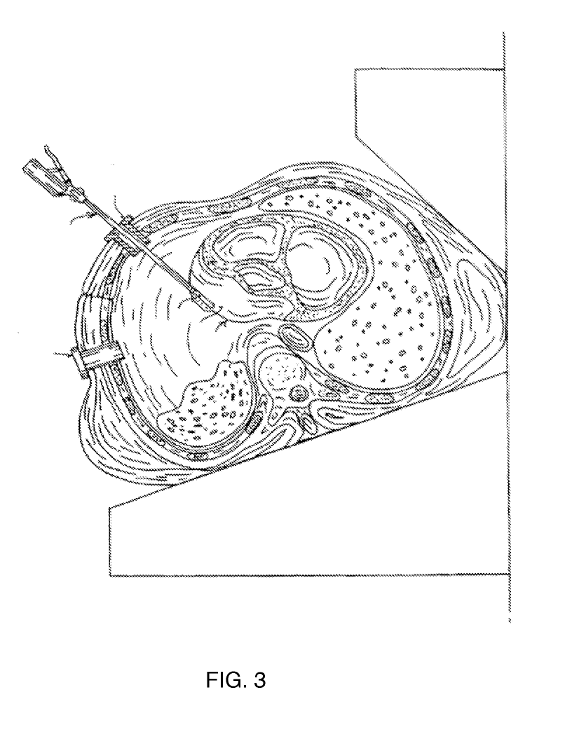

FIG. 3 is a drawing of a patient in cross-section with a catheter device shown accessing the heart via a chest wall hub. FIG. 3 shows a heart having an enlarged left ventricle 110 accessed by inserting a catheter 114 having a canula 116 and trocar 118 that is percutaneously advanced through an intercostal space and into the left ventricle 110.

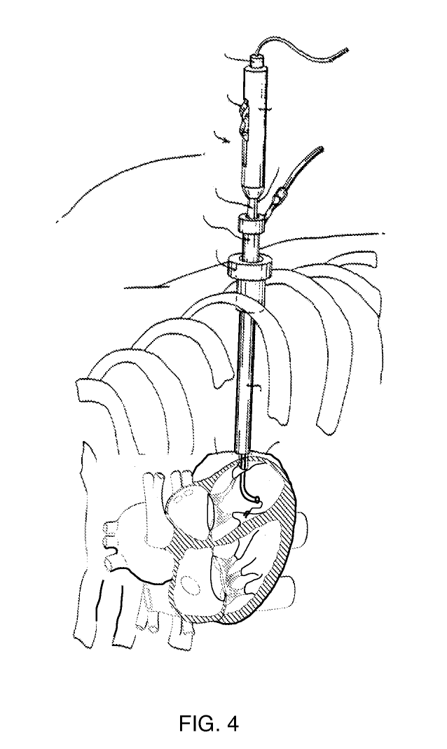

FIG. 4 is a drawing of a lateral introduction device/catheter percutaneously accessing the heart via the 5th intercostal space with catheter entering the ventricular wall and piercing the anterior papillary. Referring now to FIG. 4, once the catheter 114 reaches the interior of the left ventricle, the trocar 118 is removed in favor of a steerable guide catheter 120 which permits introduction of the instruments which will be used to engage and tether the papillary muscles, as described in more detail below. FIG. 4 shows canula and the tethering member with an attached anchor to the posterior papillary muscles.

An advantage of the left-side lateral approach is that it eliminates any risks associated with crossing the aortic valve, trans-septal puncture, or arterial damage, and permits the use of larger French catheter, and provides direct access to the papillary muscles, without requiring that the mitral valve be crossed.

Referring now to FIG. 4, the papillary muscles 210, and 220 need to be addressed using the proper orientation of the catheters, tools and the like throughout the procedure. Such orientation is accomplished using a steerable catheter 120 or equivalent tool.

In an example embodiment of the invention, the papillary muscles 210, 220 are grasped by partial or full penetration or piercing. This may be accomplished with a variety of grasping mechanisms, preferably including one or more piercing prongs extending from an instrument or catheter tool so as to grasp a target structure. Referring more specifically to the example embodiment of FIG. 4, steerable catheter 120 is fed through the guide catheter 114 to secure a first anchor 124 of a tether structure 122 to one of the papillary muscles 210 in the left ventricle.

The steerable catheter 120 is advanced from the distal end of the guide catheter 114 and may be observed in real time via any conventional imaging technique. In the illustrated example embodiment, a suture or clip applying instrument (tethering device) 122 is passed through the catheter 120. Advantageously, the instrument has a steerable tip so that it may be directed to a position in opposed facing relation to a target portion of a papillary muscle. Disposed at or adjacent the distal end of the tethering instrument 122 in this embodiment is a clamp or clip 124 for secure attachment to the respective papillary muscle. The clip or clamp is advanced out of the deployment catheter and into engagement with respective papillary muscle. Any suitable mechanism can be sued to close the clip. If deemed necessary or desirable, one or more additional clips with tethers may be applied.

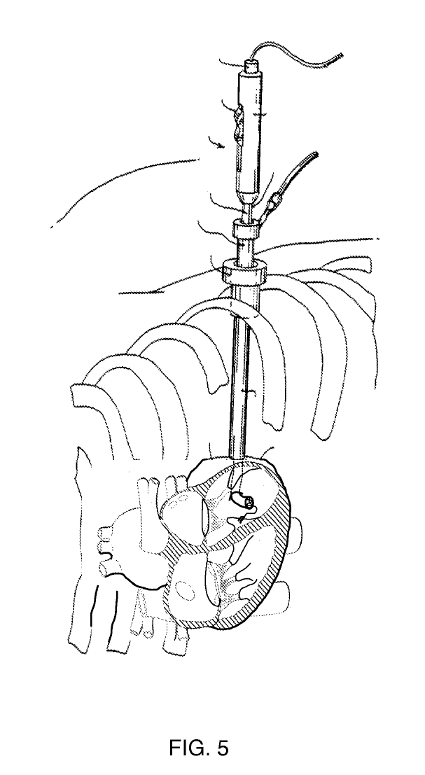

FIG. 5 is a drawing of a lateral introduction device/catheter percutaneously accessing the heart via the 5th intercostal space with catheter entering through the ventricular wall without piercing the anterior papillary. FIG. 5 shows a tethered anchor attached to the posterior papillary muscle.

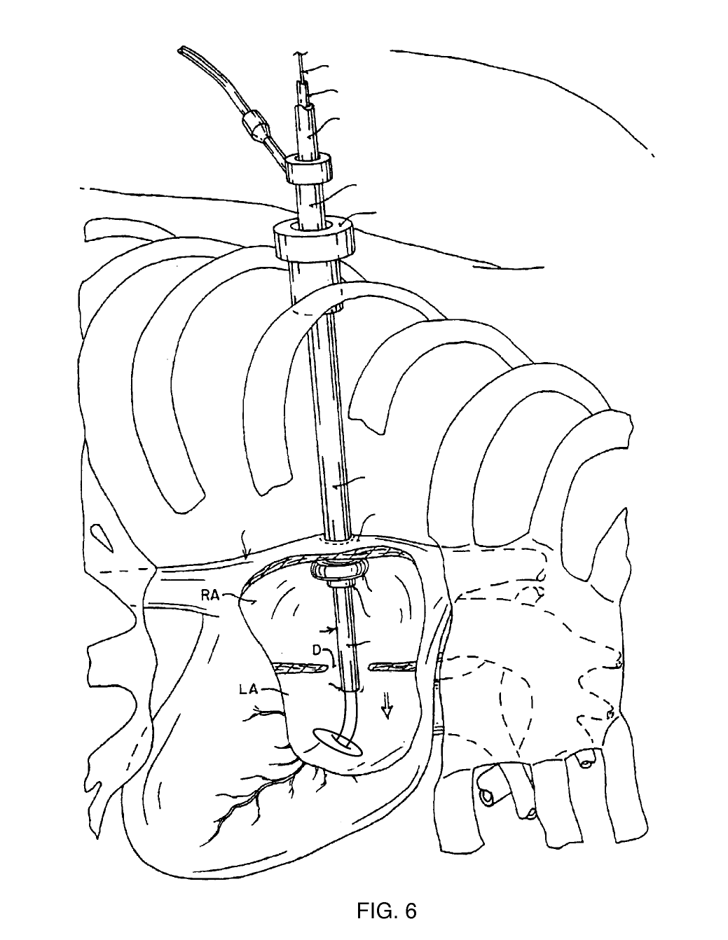

FIG. 6 is a drawing of a lateral introduction device/catheter percutaneously accessing the left ventricle of the heart via the 5th intercostal space of a patient's right side with catheter entering through a trans-septal opening from the right atrium to the left atrium for a trans-septal approach. Steerable catheter 310 is shown accessing the left atrium 320 through trans-septal aperture 330 from the right atrium 340. Steerable catheter 310 is shown positioned through mitral valve 350 to access the inside of left ventricle 360.

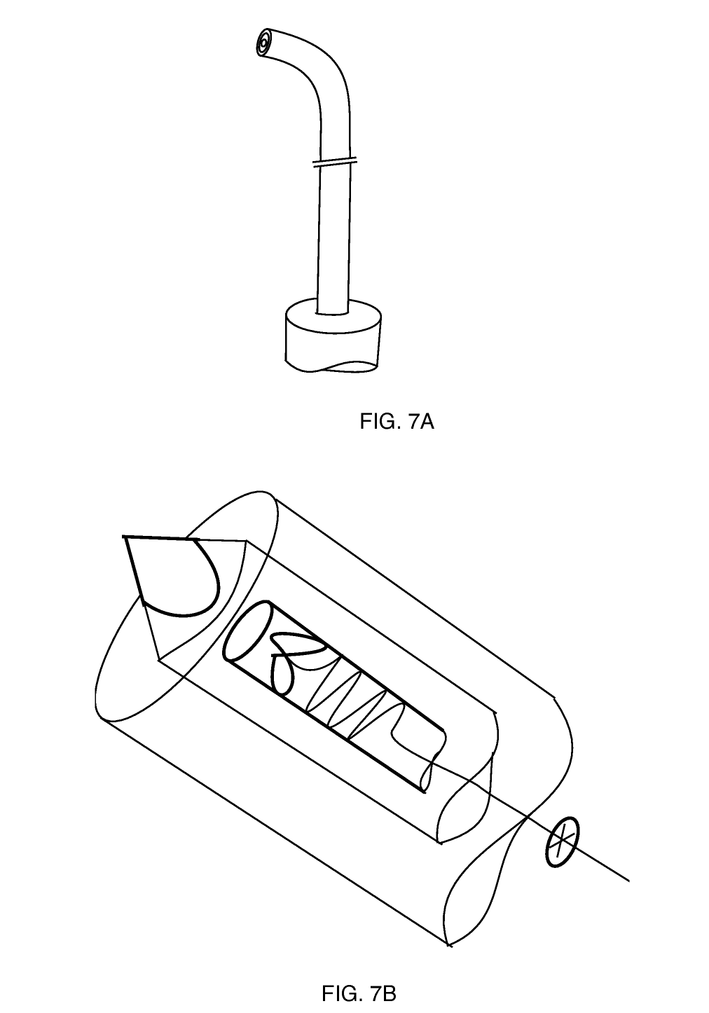

FIG. 7A is a drawing illustrating a three-shafted percutaneous access instrument. FIG. 7A shows a tool, preferably about 25 cm long and having a unique curved tip. The last 6 cm of the 25 cm length is designed with a 75 degree deflection from normal. This feature provide improved usability and access without damaging surrounding tissues. Blunt end allows for an easy introduction by dilation without significant tissue expansion damage. The tip is optionally configured to be removeable and optionally may be configured to be reuseable. It is contemplated that lengths may vary according to use, but ranges from about 20 to about 30 cm are contemplated, and deflections from about 60 degrees to about 90 degrees, and more preferably from about 70 degrees to about 80 degrees, are contemplated as within the scope of the present invention. FIG. 7B is a drawing illustrating a tip of the tethering tool and shows an external canula with a spring-loaded pointed obdurator sheathed inside of it, and an inner sleeve containing a butterfly anchor and tether, which is optionally coiled to assist installation of the tethered anchor.

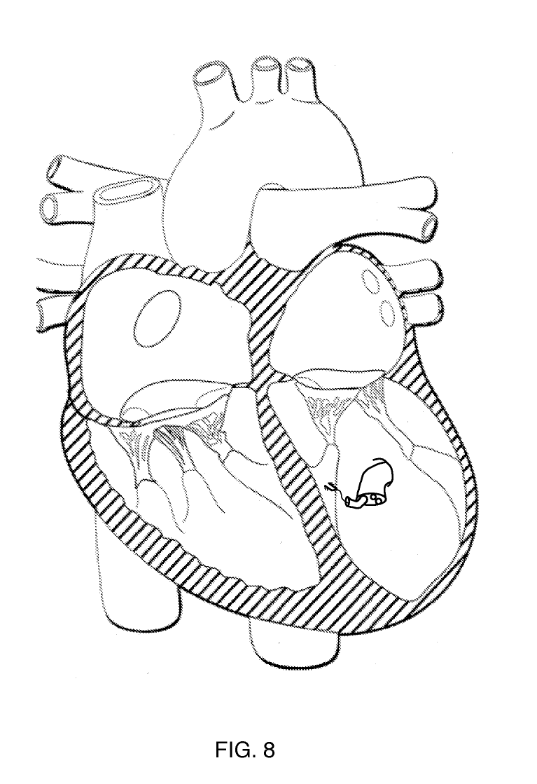

FIG. 8 is a drawing of a heart and left ventricle having a tethered anchor implanted in the posterior papillary muscle.

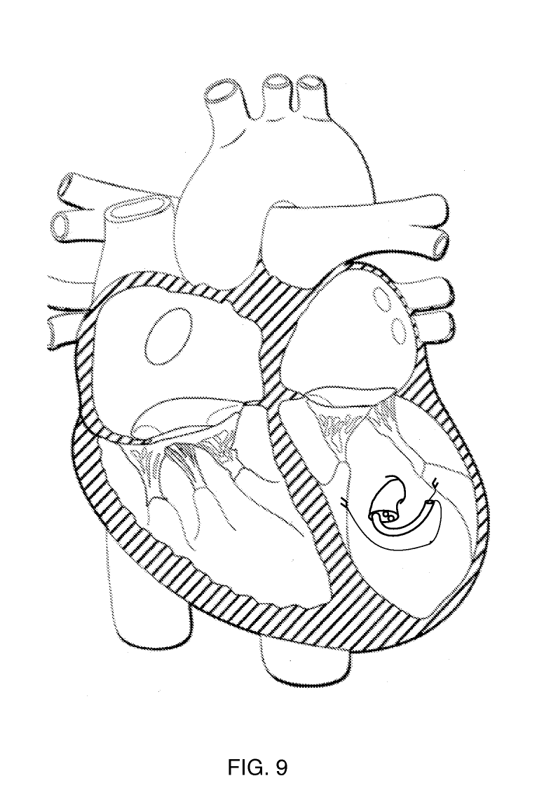

FIG. 9 is a drawing of a heart showing both a tethered anchor implanted in the anterior papillary muscle and a tethered anchor previously anchored in the posterior papillary muscle resulting in two papillary muscles anchored with an unadjusted, or loose, tether.

Referring now to FIG. 9, once the clip has been secured with respect to a first one of the papillary muscles 210, the instrument is withdrawn to reveal the flexible strand and the same or another instrument carrying another clip is conducted through the guide catheter adjacent the already placed flexible strand. In the alternative, the instrument carries at least first and second clips and respective flexible strands so that the papillary muscles can be respectively engaged without withdrawing the instrument and reinserting it. Whether the clips are attached sequentially by the sequential feed of an instrument or sequentially by manipulating the instrument, after each papillary muscle has been engaged by respective clip(s) with respective flexible strand(s), the instrument is withdrawn through the guide catheter.

According to an alternate embodiment, non-absorbable suture loop(s) may be applied directly in the papillary muscles. For example, a variation of the Perclose A-T® vasculature closure device, which is a stitch knot transmitting device with a suture cutter could be used apply a suture loop. There are also known laparoscopic devices, such as the Quik-Stitch Endoscopic Suturing System, that may be adapted to transvascularly securing a tether to the papillary muscles.

As illustrated in FIG. 9, the guide catheter 120 remains in place with the flexible tether strand(s) 126 extending therethrough from the respective secured clip/anchor 124 on first papillary muscle 210. Then, steerable catheter 120 attaches second anchor 128 to second papillary muscle 220.

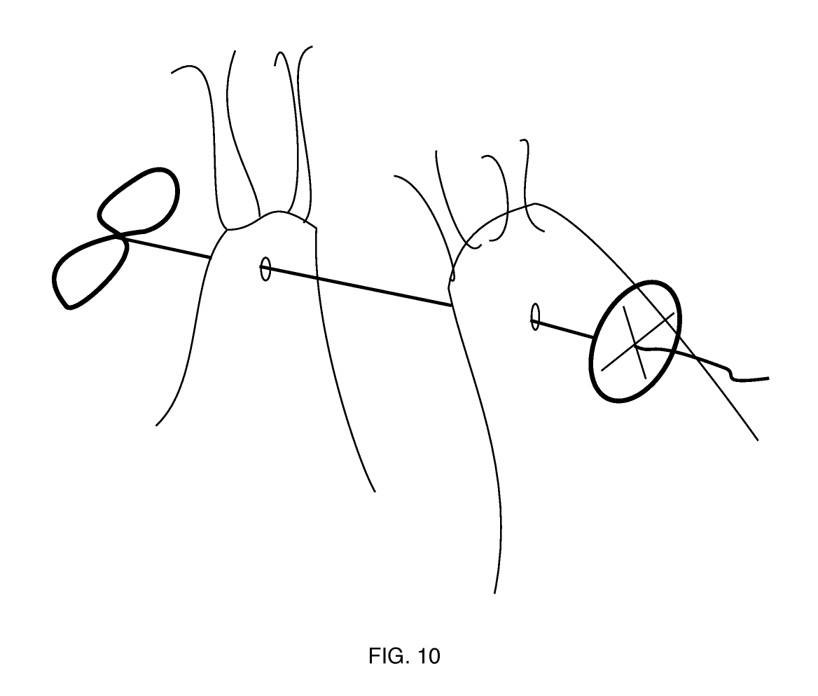

FIG. 10 is a drawing of an anterior and posterior papillary muscle having a tether inserted through an opening in the muscle tissue, and shows a figure-8, or butterfly, anchor attached to the end of the tether and located on the distal side of the posterior papillary and a cinching disk attached to the tether and located on the proximal side of the anterior papillary.

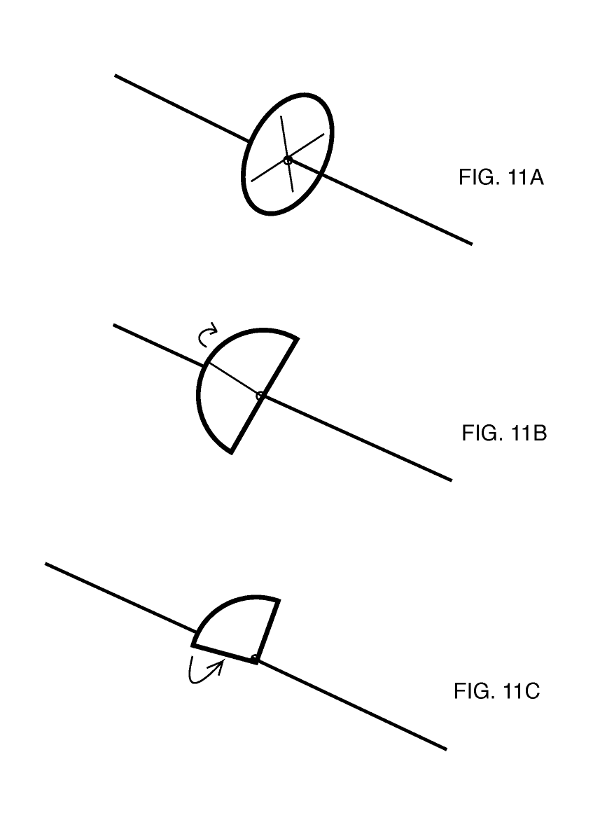

FIG. 11A is a drawing of a metallic cinch, or cinching, disk attached to a tether, where the disk has an aperture through which the tether runs, and shows Step 1 of a process of cinching, or clamping the cinch disk onto the tether at a specific location of the tether equal to a desired length of tether. FIG. 11B is a drawing of the cinch disk on the tether and shows Step 2 of the cinching process where the cinch disk is crimped or folded 180 degrees. FIG. 11C is a drawing of the cinch disk on the tether and shows final Step 3 of the cinching process where the halfed-cinch disk is again crimped or folded a second 180 degrees created a locked cinch disk, wedge shaped from the crimping, locked in place at that location on the tether.

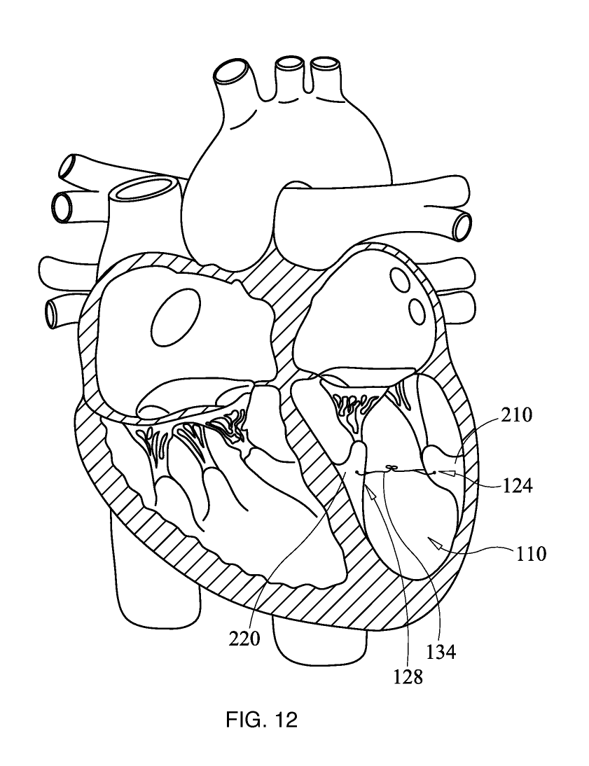

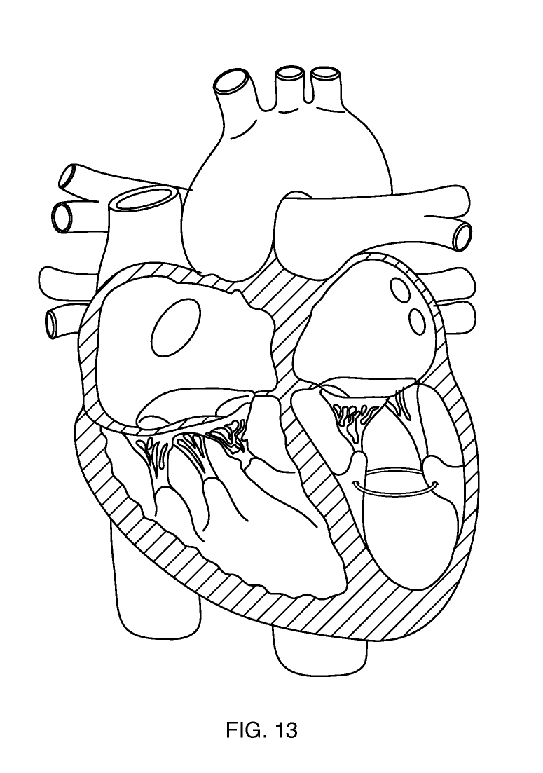

FIG. 12 is a drawing of a corrected heart showing the tethers gathered by an adjustable connector. FIG. 13 is a drawing of a corrected heart showing a circular tether embodiment.

FIGS. 12 and 13 show corrected left ventricle 110 having papillary 210 held by anchor 124, and papillary 220 held by anchor 128, and joined by connector 134, which may be adjustable. Any suitable instrument may be used to capture and sever the excess tether length such as, for example, a suture trimmer. FIG. 10 shows the tethers being cinched. Anterior papillary 210 and posterior papillary muscle 220 are shown tethered by knotted and/or adjustable tether 134. Anterior anchor 124 and and posterior anchor 128 are shown attached to their respective papillary muscles. FIG. 13 is a drawing of a heart showing a circular tether embodiment.

Referring now to FIGS. 10, 12, and 13, the tethered papillary muscles 210, 220 are tethered by tether strand 126 and 130. The tether strands 126 and 130 are next drawn together by using a gathering instrument 132, which is advanced over the flexible tethers and the tethers are pulled through the instrument to draw the clips 124, 128 toward one another. The tethers are then either tied or fastened together to define the desired spacing of the papillary muscles. For example, two tethers may have a knot transmitted to define the junction, or they are clipped to one another through the existing guiding catheter.

The tethering and drawing of the papillary muscles towards one another may be conducted while monitoring the position of the muscles fluoroscopically, and under intra-cardiac ultrasound guidance, so that the papillary muscles can be drawn to a desired transventricular distance. Intra cardiac Echo Doppler can also be used to assess the severity of LV enlargement/CV disease, or regurgitation, to adjust the length of the tethers to an optimum transventricular distance to suppress cardiac deficiency or regurgitation. So bringing the papillary muscles closer together reduces the size of the left ventricular cavity and will limit further distension of the ventricular wall, thereby mimicking the effect of the congenital false tendon to improve ventricular geometry and mitigate the effects of Dilated Cardiomyopathy.

### Example Technique

In one preferred technique, the method comprises the steps of piercing the posterior papillary muscle, extending the catheter to install one or more tethered anchors to the anterior papillary muscle, withdrawing the catheter to install one or more tethered anchors onto the posterior papillary muscle, and then cinching the tethers together to achieve the desired geometry of the left ventricle and/or the mitral valve.

In another variation of this technique, the posterior papillary is pierced, and the anterior papillary is captured by looping one or more tethers around it, the tether(s) is/are tightened to cinch the papillary muscles are cinched without use of anchors, or with minimal use of anchors only on the posterior papillary.

### Example Technique

In yet another preferred embodiment of the invention, the method comprises the steps of piercing the cardiac septum, extending the catheter to install one or more tethered anchors to the posterior papillary muscle, withdrawing the catheter to install one or more tethered anchors onto the septum, then cinching the tethers together to achieve the desired geometry of the left ventricle and/or the mitral valve.

In a variation of this technique, the septum is pierced, and the posterior papillary is captured by looping one or more tethers around it, the tether(s) is/are tightened to cinch the papillary muscle without use of anchors, or with minimal use of anchors only on the septum.

### Example Technique

In yet another preferred embodiment of the invention, the method comprises the steps of piercing the left ventricular wall, extending the catheter to install one or more tethered anchors to the posterior and/or anterior papillary muscle, withdrawing the catheter to install one or more tethered anchors onto the left ventricular wall, then cinching the tethers together to achieve the desired geometry of the left ventricle and/or the mitral valve.

In a variation of this technique, the left ventricular wall is pierced, and the posterior and/or anterior papillary is captured by looping one or more tethers around it, the tether(s) is/are tightened to cinch the papillary muscle(s) without use of anchors, or with minimal use of anchors only on the left ventricular wall.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A method for improving cardiac function by reducing ventricular volume, comprising the steps of: inserting by percutaneous approach a tether installation device into a patient; and inserting said tether installation device through the lateral wall of the left ventricle of the patient's heart; attaching at least one tethered papillary muscle anchor from within said tether installation device to a papillary muscle within said left ventricle; withdrawing said tether installation device from said left ventricle such that the tether of the tethered papillary muscle anchor tranverses the left ventricular wall and extends from the inside to the outside of the left ventricle; attaching a pledget to the tether portion outside the left ventricle to form a wall anchor; wherein said papillary anchor and said wall anchor are joined by the tether to change the geometry and reduce the volume of the left ventricle.

2. The method as claimed in claim 1, further comprising the step of adjusting the tether member to achieve a desired geometry of the left ventricle.

3. The method as claimed in claim 1, further comprising the steps of attaching at least one additional papillary muscle anchor joined by an additional tether member so as to achieve a desired geometry of the left ventricle.

4. The method as claimed in claim 1, further comprising the step of adjusting the tether member to achieve coaptation of the mitral valve.

5. The method of claim 1, further comprising wherein the step of inserting by percutaneous approach further comprises using a non-coring needle.

6. The method of claim 6, further comprising wherein the needle used is a small gauge needle used as a guide to reduce the defect made to the tissue, and the opening is then temporarily dilated using the larger bore instrument to house the catheter.

7. The method of claim 1, further comprising wherein after the left ventricular wall is pierced, the posterior and/or anterior papillary is captured by looping one or more tethers around it, the tether(s) is/are tightened to cinch the papillary muscle(s) without use of anchors, or with minimal use of anchors only on the left ventricular wall.

8. A method for improving cardiac function by reducing ventricular volume, comprising the steps of: inserting by percutaneous approach a tether installation device into a patient; and inserting said tether installation device through the lateral wall of the left ventricle of the patient's heart and into the left ventricle of the patient's heart; and attaching a first tethered papillary muscle anchor from within said tether installation device to the posterior papillary muscle within said left ventricle; attaching a second tethered papillary muscle anchor from within said tether installation device to the anterior papillary muscle of the left ventricle of the patient's heart; withdrawing said tether installation device from said left ventricle such that the tethers of the tethered papillary muscle anchors tranverse the left ventricular wall and extend from the inside to the outside of the left ventricle; attaching one or more pledgets to the tether portions outside the left ventricle to form one or more wall anchors; wherein said papillary anchors and said wall anchor are joined by the tethers to change the geometry and reduce the volume of the left ventricle.

9. The method as claimed in claim 8, further comprising the step of adjusting the tether member to achieve a desired geometry of the left ventricle.

10. The method as claimed in claim 8, further comprising the steps of attaching at least one additional papillary muscle anchor joined by an additional tether member so as to achieve a desired geometry of the left ventricle.

11. A method for improving cardiac function, comprising the steps of: inserting by percutaneous approach a tether installation device into a patient, said tether installation device having a catheter for advancing one or more tethers, tethered anchors, and endoscopic tools; and inserting said tether installation device through the lateral wall of the left ventricle of the patient's heart; piercing the posterior papillary muscle, extending the catheter to install one or more tethered anchors to the anterior papillary muscle, withdrawing the catheter to install one or more tethered anchors onto the posterior papillary muscle, and then cinching the tethers together to achieve the desired geometry of the left ventricle and/or the mitral valve.

12. The method of claim 11, further comprising wherein after the posterior papillary is pierced, the anterior papillary is captured by looping one or more tethers around it, the tether(s) is/are tightened to cinch the papillary muscles are cinched without use of anchors, or with minimal use of anchors only on the posterior papillary.

13. A method for improving cardiac function, comprising the steps of: inserting by percutaneous approach a tether installation device into a patient, said tether installation device having a catheter for advancing one or more tethers, tethered anchors, and endoscopic tools; and inserting said tether installation device through the lateral wall of the left ventricle of the patient's heart; piercing the cardiac septum, extending the catheter to install one or more tethered anchors to the posterior papillary muscle, withdrawing the catheter to install one or more tethered anchors onto the septum, then cinching the tethers together to achieve the desired geometry of the left ventricle and/or the mitral valve.

14. The method of claim 13, further comprising wherein after the septum is pierced, and the posterior papillary is captured by looping one or more tethers around it, the tether(s) is/are tightened to cinch the papillary muscle without using anchors, or with minimal use of anchors only on the septum.

15. A method for improving cardiac function, comprising the steps of: inserting by percutaneous approach a tether installation device into a patient, said tether installation device having a catheter for advancing one or more tethers, tethered anchors, and endoscopic tools; and inserting said tether installation device through the lateral wall of the left ventricle of the patient's heart; extending the catheter to install one or more tethered anchors to the posterior and/or anterior papillary muscle, withdrawing the catheter to install one or more tethered anchors onto the left ventricular wall, then cinching the tethers together to achieve the desired geometry of the left ventricle and/or the mitral valve.

16. The method as claimed in claim 15, further comprising the step of adjusting the tether member to achieve coaptation of the mitral valve.

17. The method of claim 15, further comprising wherein the step of inserting by percutaneous approach further comprises using a non-coring needle.

18. The method of claim 15, further comprising wherein the needle used is a small gauge needle used as a guide to reduce the defect made to the tissue, and the opening is then temporarily dilated using the larger bore instrument to house the catheter.

19. The method of claim 15, further comprising implanting a valve at the lateral wall insertion site on the heart of the patient, wherein said valve is a blood leakage control valve/sleeve.

20. A medical device for improving cardiac function or reducing ventricular volume, comprising: a canula having a tethering device disposed therein; said canula having a trocar or needle-like device for piercing the lateral wall of the left ventricle of the patient's heart; said tethering device comprising at least one first papillary muscle anchor for attaching to a first papillary muscle within said left ventricle and at least one second papillary muscle anchor for attaching to a second papillary muscle of the patient's heart; said tethering device further comprising a tether member for joining said first papillary muscle anchor to said second papillary muscle anchor so as to reduce the left ventricular volume of the patient.

21. The device of claim 20, wherein said tether member has an adjustable mechanism for adjusting the length of said tether.

22. The device of claim 20, further comprising at least one additional papillary muscle anchor joined by an additional tether member so as to achieve a desired geometry of the left ventricle.

23. The device of claim 20, wherein the tether member is comprised of nitinol (nickel-titanium shape memory alloy) or austinetic stainless steel.

24. The medical device of claim 20, further comprising wherein the needle-like device is a non-coring needle.

25. The medical device of claim 20, further comprising wherein the needle used is a small gauge needle used as a guide to reduce the defect made to the tissue, and the opening is then temporarily dilated using the larger bore instrument to house the catheter.

26. The medical device of claim 20, further comprising a leakage control valve/sleeve operatively associated with the canula that allows access to the interior of the left ventricle and controls blood loss during tethering.
